(19) United States
(12) Patent Application Publication
(10) Pub. No.: US 2010/0113783 A1
(43) Pub. Date: May 6, 2010

(54) PROCESS FOR THE PREPARATION OF CRYSTALS OF PRULIFLOXACIN

(75) Inventors: Tarun Kant Sharma, Gurgaon (IN); Sandeep T. Kanawade, Pune/Maharastra (IN)

Correspondence Address: Ranbaxy Inc., Intellectual Property Department, 600 College Road East, PRINCETON, NJ, 08540, US

(73) Assignee: RANBAXY LABORATORIES LIMITED, GURGAON (IN)

(21) Appl. No.: 12/531,243

(22) PCT Filed: Mar. 14, 2008
(86) PCT No.: PCT/IB08/50974

§ 371 Date: Jan. 21, 2010

(30) Foreign Application Priority Data
Mar. 14, 2007 (IN) 544/DEL/2007

(51) Int. Cl. C07D 417/14

(52) U.S. Cl. 544/361

(57) ABSTRACT

The present invention relates to processes for the preparation of Type I, Type II and Type III crystals of prulifloxacin.

### FIELD OF THE INVENTION

The present invention relates to processes for the preparation of Type I, Type II and Type III crystals of prulifloxacin.

### BACKGROUND OF THE INVENTION

Prulifloxacin is chemically 6-fluoro-1-methyl-7-{4-[(5-methyl-2-oxo-1,3-dioxol-4-yl)methyl]piperazin-1-yl}-4-oxo-4H-[1,3]thiazeto[3,2-α]quinoline-3-carboxylic acid of Formula I having the structure as depicted below:

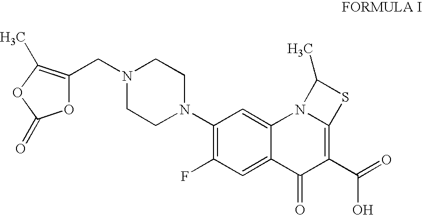

Prulifloxacin has significant antibacterial activity and has been marketed as a synthetic antibacterial agent. U.S. Pat. No. 5,086,049 provides a process for the preparation of prulifloxacin, wherein the final prulifloxacin is recrystallized from chloroform-methanol mixture.

EP Patent No. 1,626,051 A1 mentions that Type I, Type II and Type III crystals of prulifloxacin are obtained by crystallization from acetonitrile as reported in Iyakuhin Kenkyu, Vol. 28 (1), (1997), 1-11. However, this publication mentions that the conditions of crystallization from acetonitrile for preparing Type I, Type II and Type III crystals are not disclosed in Iyakuhin Kenkyu, Vol. 28 (1), (1997), 1-11. EP 1,626,051 further mentions that Type III crystals have been marketed by considering the solubility, absorbability, therapeutic effect and the like of the respective crystal forms.

EP 1,626,051 provides processes for the preparation of Type I and Type II crystals from acetonitrile by the addition of their respective seed crystals, but does not disclose the means for preparation of seed crystals. According to EP 1,626,051, when prulifloxacin is crystallized from acetonitrile without adding any seed crystal, it results in the formation of an acetonitrile solvate of prulifloxacin, referred to as ‘Compound B’. Type III crystals are reportedly obtained when Compound B is desolvated. EP 1,626,051 states that Compound B needs to be prepared as an intermediate for producing Type III crystals and Type III crystals as such are not directly obtainable by crystallization from acetonitrile even if seeding is performed with Type III crystals. This application provides various processes for the preparation of Compound B of prulifloxacin from acetonitrile by controlling supersaturation concentration at the time of spontaneous nucleation or at the addition of Compound B seed crystals. Thus, the process provided in EP 1,626,051 for the preparation of Type III crystals is complex, in that it requires the preparation Compound B and the conversion of Compound B into Type III crystals, and the preparation of Compound B involves critical monitoring of supersaturation concentration at specific ranges.

### SUMMARY OF THE INVENTION

The present inventors have developed simple methods for the preparation of Type I, Type II and Type III crystals using acetonitrile. The present process does not involve the addition of seed crystals at any stage. Further, the present inventors have surprisingly found that Type III crystals can be obtained directly by crystallization from acetonitrile without involving the preparation and desolvation of an acetonitrile solvate. By employing the present invention, any solid form prulifloxacin can be converted into Type I, Type II or Type III crystal forms. Thus, the present invention provides simple, efficient and industrially applicable processes for the preparation of Type I, Type II and Type III crystals of prulifloxacin.

### DETAILED DESCRIPTION OF THE INVENTION

In a first aspect, a process for the preparation of Type I crystals of prulifloxacin is provided, wherein the process comprises,
a) dissolving prulifloxacin in acetonitrile by heating to a temperature of about 75° C. or more,
b) cooling the solution obtained in step a) to a temperature of about 25° C. to about 35° C. in about 2 hours or more,
c) isolating Type I crystals of prulifloxacin.

Prulifloxacin of any solid form can be used as a starting material. Prulifloxacin can be prepared, for example, according to methods, for example those provided in U.S. Pat. No. 5,086,049 or EP 1,626,051 A1. The prulifloxacin can be dissolved in acetonitrile by heating to a temperature of about 75° C. or more. The solution so obtained is cooled to a temperature of about 25° C. to about 35° C. in about 2 hours or more, preferably in about 7 hours to about 9 hours. After the formation of crystals, the reaction mixture is optionally further cooled to about 0° C. to about 10° C. accompanied by stirring. The crystals are subsequently dried to obtain Type I prulifloxacin.

In a second aspect, a process for the preparation of Type II crystals of prulifloxacin is provided, wherein the process comprises,
a) dissolving prulifloxacin in acetonitrile by heating to a temperature of about 75° C. or more,
b) cooling the solution obtained in step a) to a temperature of about 0° C. to about 10° C. in about 1 hour or less,
c) isolating Type II crystals of prulifloxacin.

Prulifloxacin of any solid form can be used as a starting material. Prulifoxacin can be prepared, for example, according to methods, for example those provided in U.S. Pat. No. 5,086,049 or EP 1,626,051 Al. The prulifloxacin can be dissolved in acetonitrile by heating to a temperature of about 75° C. or more. The solution so obtained is cooled to a temperature of about 0° C. to about 10° C. in about 1 hour or less, for example, in about 5 minutes to about 20 minutes. The mixture is stirred at this same temperature to effect maximum crystallization. The crystals are subsequently dried to obtain Type II prulifloxacin.

In a third aspect, a process for the preparation of Type III crystals of prulifloxacin is provided, wherein the process comprises,
a) dissolving prulifloxacin in acetonitrile by heating to a temperature of about 75° C. or more,
b) cooling the solution obtained in step a) to a temperature of about 25° C. to about 35° C. in about 1 hour or less,
c) isolating Type III crystals of prulifloxacin.

Prulifloxacin of any solid form can be used as a starting material. Prulifloxacin can be prepared, for example, according to methods, for example those provided in U.S. Pat. No. 5,086,049 or EP 1,626,051 A1. The prulifloxacin can be dissolved in acetonitrile by heating to a temperature of about 75° C. or more. The solution so obtained is cooled to a temperature of about 25° C. to about 35° C. in about 1 hour or less, for example in about 20 minutes to about 40 minutes. After the formation of crystals, the mixture is optionally further cooled to about 0° C. to about 10° C. accompanied by stirring. The crystals are subsequently dried to obtain Type III prulifloxacin.

### BRIEF DESCRIPTION OF THE DRAWINGS

FIG. 1 is an XRPD of Type I crystals of prulifloxacin.

FIG. 2 is an XRPD of Type II crystals of prulifloxacin.

FIG. 3 is an XRPD of Type III crystals of prulifloxacin.

Powder XRD of the samples were determined by using X-Ray Difractometer, Rigaku Corporation, RU-H3R, Goniometer CN2155A3, X-Ray tube with Cu target anode, Divergence slits 1 0, Receiving slit 0.15 mm, Scatter slit 1°, Power: 40 KV, 100 mA, Scanning speed: 2 deg/min step: 0.02 deg, Wave length: 1.5406 A.

While the present invention has been described in terms of its specific embodiments, certain modifications and equivalents will be apparent to those skilled in the art and are intended to be included within the scope of the present invention.

### EXAMPLES

### Example 1

### Process for the Preparation of Type I Crystals of Prulifloxacin

Prulifloxacin (100 g) was dissolved in acetonitrile (5.5 L) at reflux temperature. The undissolved materials were filtered out. The filtrate obtained was cooled slowly to 28° C. in 8 hours. The reaction mixture was further cooled to 5° C. and stirred for 3 hours.

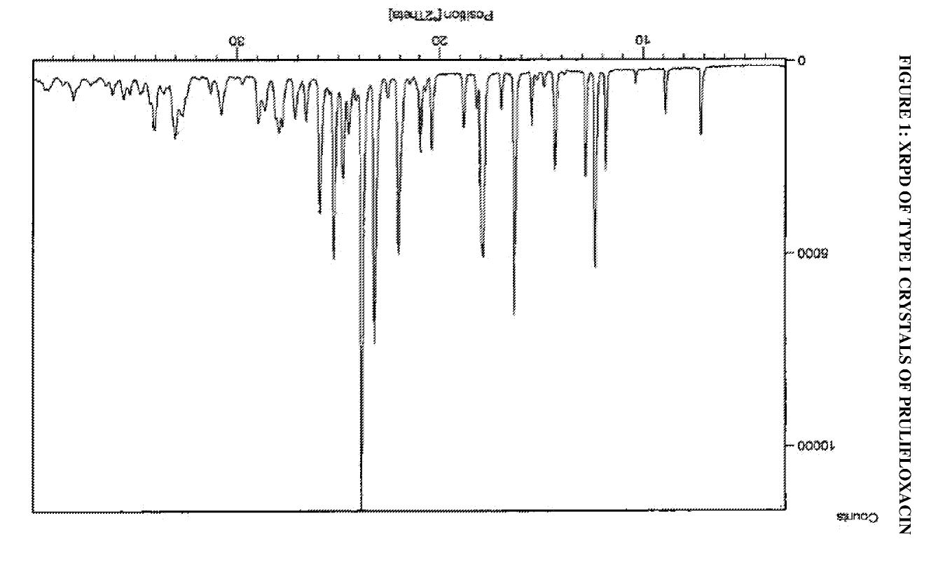

The solid obtained was dried at 60° C. for 24 hours to obtain the title compound having an XRPD pattern as depicted in FIG. 1.

Yield: 85%

### Example 2

### Process for the Preparation of Type II Crystals of Prulifloxacin

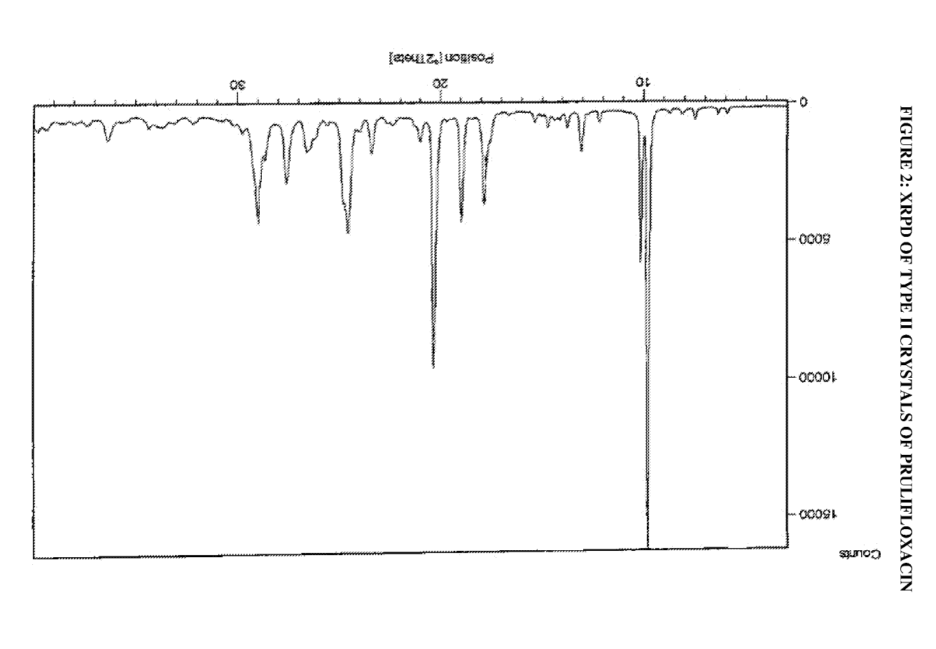

Prulifloxacin (100 g) was dissolved in acetonitrile (5.5 L) at reflux temperature. The undissolved materials were filtered out. The filtrate obtained was cooled rapidly to 5° to 7° C. in 10 minutes and stirred for 3 hours. The solid obtained was dried at 55° C. for 24 hours to obtain the title compound having an XRPD pattern as depicted in FIG. 2.

Yield: 85%

### Example 3

### Process for the Preparation of Type III Crystals of Prulifloxacin

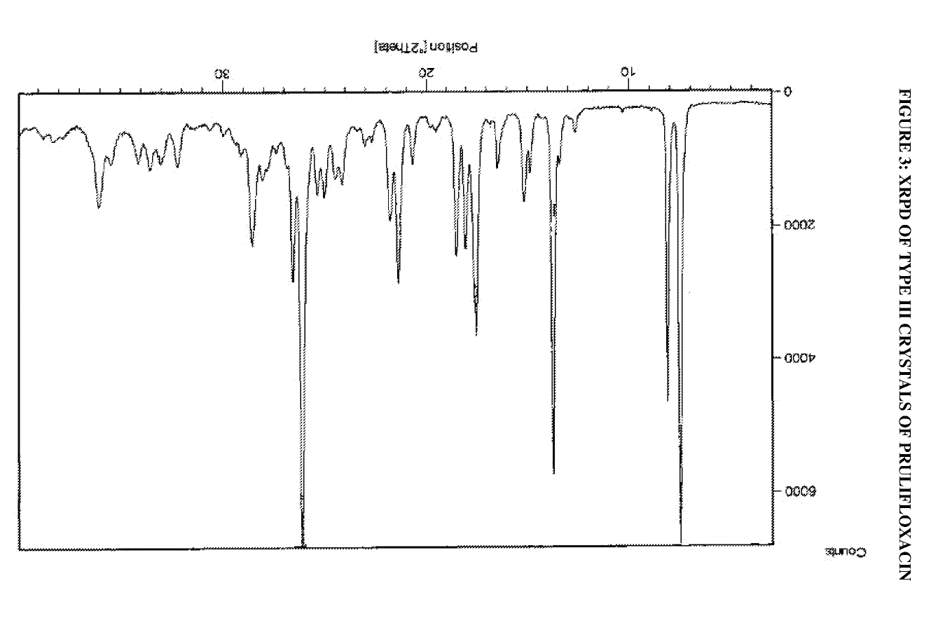

Prulifloxacin (100 g) was dissolved in acetonitrile (5.5 L) at reflux temperature. The undissolved materials were filtered out. The filtrate obtained was cooled to 28° C. in 30 minutes, and subsequently to 5° C. followed by stirring for 3 hours. The solid obtained was dried at 60° C. for 24 hours to obtain the title compound having an XRPD pattern as depicted in FIG. 3.

Yield: 85%

We claim:

1. A process for the preparation of Type I crystals of prulifloxacin, the process comprising:
a) dissolving prulifloxacin in acetonitrile by heating to a temperature of about 75° C. or more;
b) cooling the solution obtained in step a) to a temperature of about 25° C. to about 35° C. in about 2 hours or more; and
c) isolating Type I crystals of prulifloxacin.

2. A process according to claim 1, wherein cooling is carried out in about 7 hours to about 9 hours.

3. A process according to claim 1, wherein step b) further comprises cooling the mixture to about 0° C. to about 10° C.

4. A process for the preparation of Type II crystals of prulifloxacin, the process comprising:
a) dissolving prulifloxacin in acetonitrile by heating to a temperature of about 75° C. or more;
b) cooling the solution obtained in step a) to a temperature of about 0° C. to about 10° C. in about 1 hour or less; and
c) isolating Type II crystals of prulifloxacin.

5. A process according to claim 4, wherein cooling is carried out in about 5 minutes to about 20 minutes.

6. A process according to claim 4, wherein step b) further comprises stirring.

7. A process for the preparation of Type III crystals of prulifloxacin, the process comprising:
a) dissolving prulifloxacin in acetonitrile by heating to a temperature of about 75° C. or more;
b) cooling the solution obtained in step a) to a temperature of about 25° C. to about 35° C. in about 1 hour or less; and
c) isolating Type III crystals of prulifloxacin.

8. A process according to claim 7, wherein cooling is carried out in about 20 minutes to about 40 minutes.

9. A process according to claim 7, wherein step b) further comprises cooling the mixture to about 0° C. to about 10° C.

10. A process according to claim 7, wherein step b) further comprises stirring.
